(19) United States
(12) Patent Application Publication
(10) Pub. No.: US 2018/0250669 A1
(43) Pub. Date: Sep. 6, 2018

(54) DEVICE AND METHOD FOR FLUIDS SEPARATION BY DENSITY GRADIENT

(71) Applicant: SPARK-TECH SP. Z O.O., Kraków (PL)

(72) Inventors: Mateusz Grzegorz ADAMSKI, Kraków (PL); Patryk GUMANN, Kraków (PL)

(21) Appl. No.: 15/759,191

(22) PCT Filed: Sep. 15, 2016
(86) PCT No.: PCT/IB2016/055503

§ 371 Date: Mar. 9, 2018

(30) Foreign Application Priority Data
Sep. 15, 2015 (PL) P.413910
Sep. 15, 2016 (PL) P.418711

(51) Int. Cl. B01L 3/00; G01N 33/49; B01D 21/26; B01D 21/00

(52) U.S. Cl. CPC B01L 3/5021; G01N 33/491; B01D 21/26; B01D 21/0036; B01L 2400/0633; B01L 2200/0647; B01L 2300/0832; B01L 2300/0609; B01L 2300/087; B01L 2400/0409

(57) ABSTRACT

A device for a centrifugation container, such as a tube, is for separation of liquid fractions having a desired density range, in particular to biological and/or liquids forming suspensions. The device has a partition (7) that separates the interior of the container (1) into at least two chambers in a vertical arrangement—an upper chamber (2) and a lower chamber (3). The device having the partition (7) has an aperture (4) which can be lined up with the guide (12), on which liquids, in particular a fluid sample, can flow down from an upper chamber (3) to a lower chamber (4), of the container (1) for centrifugation. A method using the device separates out a fraction having the desired density range from the sample containing fractions of different density.

The invention relates to a device, a container with the device and a method for fluid separation by means of density gradient centrifugation. At the same time the invention relates to a kit for carrying out the method. In particular, the invention is used to separate body fluids e g animal blood, human blood, for further analyses like clinical diagnostics or research. This invention relates to the fields of containers for laboratory use, in particular to a specialized centrifugal tubes/containers. Another purpose of the invention relates to the area associated with testing or analyzing of materials by determining their chemical, physical or biological properties, in particular the analysis of liquid biological material, for example blood.

### STATE OF THE ART

Collection, purification, separation into fractions and/or preservation of fluid samples, including blood, play an important role in medical diagnostics as well as in clinical trials. In the case of conventional systems and methods for collecting blood samples on a large scale, a blood sample obtained from a patient can be separated into different fractions by centrifugation, filtration, or elutriation and stored for later use or further testing. The separated blood components typically include fractions of red cells, white cells, platelets and plasma. Blood separation into its fractions can be performed continuously during collection of blood or in steps after it has been collected. It is critical for a number of therapeutic applications and for purposes of clinical trials that blood separation into its various fractions takes places in a highly sterile conditions.

There are many methods for blood separation into its fractions. State-of-the-art methods require the use of high-quality specialized medical devices as well as highly trained personnel for their correct operation.

A technique is known, from the international patent application WO8805331, to separate white blood cells (leukocytes) from red blood cells (erythrocytes). It involves mixing a blood sample with a working solution which then aggregates the red blood cells, as a result the sedimentation rate of agglutinated red blood cells increases. The density of the separation fluids is adjusted such that the sedimentation process of white blood cells is only slightly altered. This prevents the sedimentation of the white blood cells on bottom of container, after separation white blood cells can be collected from the upper portion of the separated blood sample, while at the same time red blood cells sediment to the bottom of the container.

Yet another technique, where the working solution for aggregation of the red blood cells is not mixed with blood, blood sample is carefully layered onto a surface of working separation fluids. As a result, the red blood cells start to agglutinate or aggregate on the interface surface between blood and separation liquids and sink to the bottom of the tube. There are several well-known polymer-based compounds which cause agglutination of red blood cells, for example. FICOLL 400 (Pharmacia Fine Chemicals, Sweden). Separation of the blood can take place under the influence of gravity or under the influence of the centrifugation. As an effect of separation, majority of white blood cells remains at the liquid interface. However, this methods cannot separate white blood cells to subpopulations, e.g. to peripheral blood mononuclear cells (PBMC) and to polymorphonuclear cells (PMN). The most needed method would consist of a one-step process where a separation medium has density that allows separating white blood cells into subpopulations simultaneously.

In order to separate subpopulations of white blood cells, one of the known methods for isolation of mononuclear white blood cells (PBMC) employs density gradient centrifugation. In the first step of this method, a mixture of Isopaque-Ficoll (Nyegaard & Co., Norway) with metrizoat as a main component, is being used. The second step of this method enables isolation of PMN fraction from blood employing dextran or gelatin, which causes increased sedimentation of red blood cells. Another method uses a discontinuous density gradients where two or more working fluids are carefully layered on top of each other. Densities are chosen such that the noncontinuous gradient is in the optimal required range—it is being chosen according to the density of separated substance.

Yet another U.S. Pat. No. 4,824,560 A discloses methods and means of rotation of the tubular container having at least two adjacent chambers which are connected to each other by a narrow, capillary-like opening. Operation principles are as following: the working fluid is placed in the lower chamber, and the fluid to be separated into fractions is applied in the upper chamber. There is no need for any special precautions to avoid mixing of the fluids before centrifugation. This method has several advantages over the manual methods described above. It also possess a disadvantage because the narrow opening between the two chambers prevents efficient passage of blood cells between the two chambers, even during centrifugation, as a result the efficiency of the blood separation is reduced.

Significant difficulty in described above manual separation methods is mainly in the sample preparation, in particular the layering of working fluids used for separation of fluid sample of different density, for example blood. It is essential for this methods that liquids of different density do not mix with each other and are separated by clear interface between them. In order to properly achieve these conditions a various techniques have been developed. An adequate and careful preparation and layering of the liquids, one on top of the another, used for the blood separation is one of them, mainly done by very careful pipetting of the liquids into the container for further separation into fractions by means of centrifugation (in order to obtain density gradient). Unfortunately, all of these procedures are cumbersome, difficult to perform, can introduce the possibility of random human errors, and in addition require highly qualified personnel, what entails high maintenance costs, reduces the reproducibility of the procedure and makes it impossible to carry out separations on a large scale.

The aim of the embodiments of the present invention is to provide a tool for the rapid and partly automated separation of fluids into fractions of various densities like in case of biological fluids, including blood, which also may allow for purification, isolation and preservation of biological samples.

Definitions

The following terms will be used in the text of the description of the invention and the claims:
“Container” includes any receptacle for collecting liquid, which is adapted to use in centrifuges, for example centrifugal tubes.
“Guide” is a part of the device which controls fluid flow speed and direction while flowing from the upper chamber to the lower chamber through the opening in the partition disk, wherein the guide should have an adequate size, to allow flow and layering of one liquid from the upper chamber on top of the other liquid in the lower chamber in particular the fluid sample on the separation fluid/medium already located at the bottom of the container. Guide in accordance to this invention can be a container wall or other structure within the container e.g. a spiral elongated sleeve, etc.

### DETAILED DESCRIPTION OF THE INVENTION

The invention relates to a device for a centrifugation container, particularly to a tube, for separation of liquid fractions having a desired density range, in particular invention applies to biological and/or liquids forming suspensions, characterized by the device having a partition that separates the interior of the container into at least two chambers in a vertical arrangement—an upper chamber and a lower chamber, and the device having the partition has an aperture which can be lined up with the guide, on which liquids, in particular fluid sample, can flow down from upper chamber to lower chamber, of the container for centrifugation.

In preferred embodiment of this invention, the guide is the inner wall of centrifuge container, a spiral, funnel or vertical elements in the shape of an elongated cylinder.

Preferably, the partition disk consists of two adjacent surfaces with apertures, in particular in the shape of flattened disks fitted to a container having a cross section similar to the wheel, where the surfaces are movable with respect to each other and their positioning relative to each other can be adjusted allowing for closing communication via partition apertures.

Preferably, the upper chamber additionally have a vertical partition or partitions dividing it into sub-chambers, each of the sub-chambers having an aperture.

In another aspect, the invention relates to a container for centrifugation comprising device for centrifugation container, particularly for a tube, for separation of liquid samples having a desired density range, particularly liquid forming a suspension or biological fluids, the device has a partition that separates the interior of the container into an upper chamber and a lower chamber, and the partition has an aperture, and near the aperture there is a guide along which the down-flow of liquids takes place, especially separation liquids flow to the lower chamber of the centrifugation container.

The partition has a aperture where a guide is placed close by along which the down-flow of liquids takes place, especially separation liquids flow to the lower chamber of the centrifugation container.

The invention also includes the method for separating out a fraction having the desired density range from the sample containing fractions of different density, especially from a biological sample, comprising:
a) providing a container with the device for the centrifuge container, in particular for a tube, for the separation of liquid sample to fractions having a desired density range by density gradient centrifugation, particularly liquid being suspensions or biological fluids,
b) filling the lower chamber of the container with medium for density gradient separation, or the upper container chamber is filled with the medium, which then through a aperture in the partition flows down on the guide to the lower chamber;
c) pouring the fluid sample (designated to be separated to fractions of different densities) into the lower chamber, by filling the upper chamber or at least one upper sub-chambers or by attaching the upper chamber to the device partition so that fluid sample can flow-down to the lower chamber through the aperture in the device partition and then along the guide and layer on the surface of separation liquids already present in the lower chamber (maintaining the interphase between liquids).
d) centrifuging the separation container until the sample separates into fractions of different density.

Preferably, step (b) is followed by an additional step or steps of (b) which entails addition of an additional medium for density gradient separation, additional media are added in the order from highest to lowest density.

Yet preferably, after step (d) selected fractions of different density from separated liquid sample can be studied, tested and analyzed, these fractions can also be preserved by freezing.

Preferably, in case of separating blood to fractions of different density, each separated fraction (each with different density) contains different blood elements including: leukocytes (lymphocytes and granulocytes), platelets, erythrocytes, bone marrow cells (megakaryocytes, erythroblasts), cells suspended in homogenate including endothelial cells, neurons, fungus, viruses, microparticles including exosomes, cellular fragments, cell organelles including nuclei, mitochondria, chloroplasts.

The invention also relates to a kit comprising:
a) the device for the container for centrifugation, in particular for tubes for separation of liquid sample to fractions of different density by density centrifugation, particularly liquids forming a suspension or biological fluids, whereas device has a partition dividing the interior of the container to the upper chamber and a lower compartment, wherein the partition has an aperture, the aperture ‘s guide, along which fluids flow-down, in particular liquid sample, to the lower chamber of the container for centrifugation
b) at least one medium for density gradient separation.

For a better understanding presented figures illustrate several embodiments of this invention. Presented illustrations do not show all possible embodiments of the invention therefore this invention cannot be limited to solutions presented in illustrations. Illustrations present:

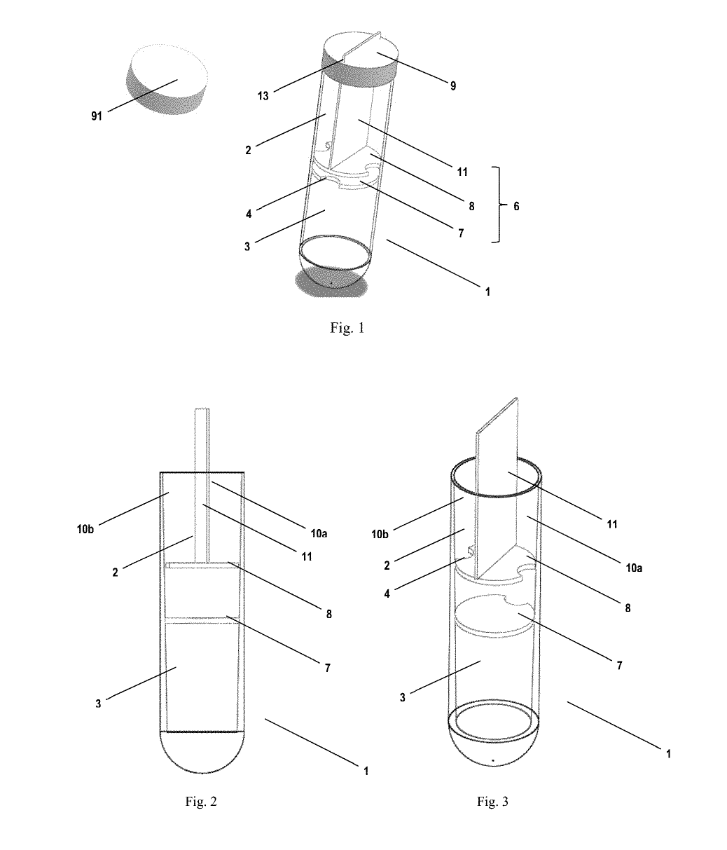

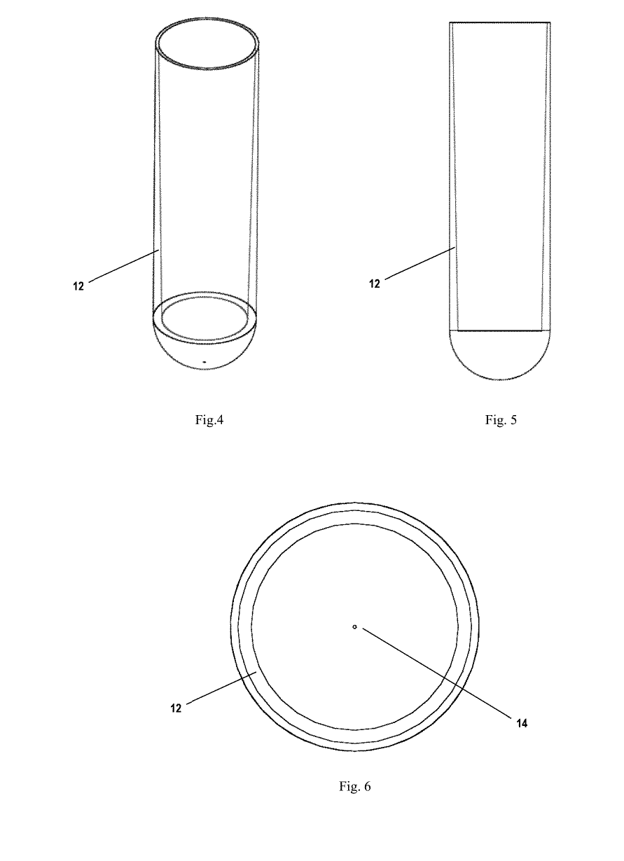

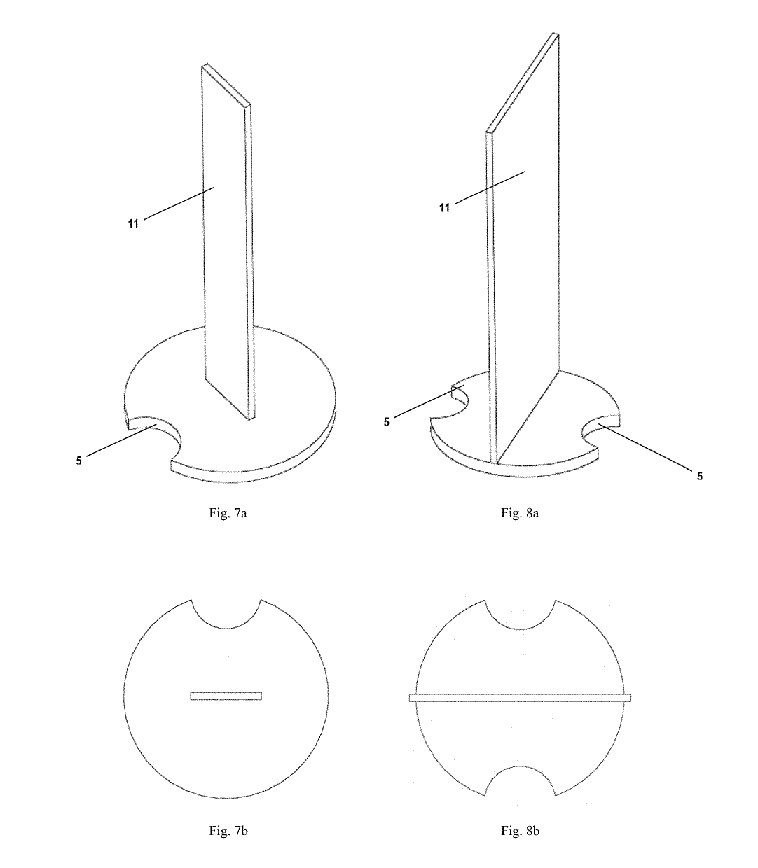

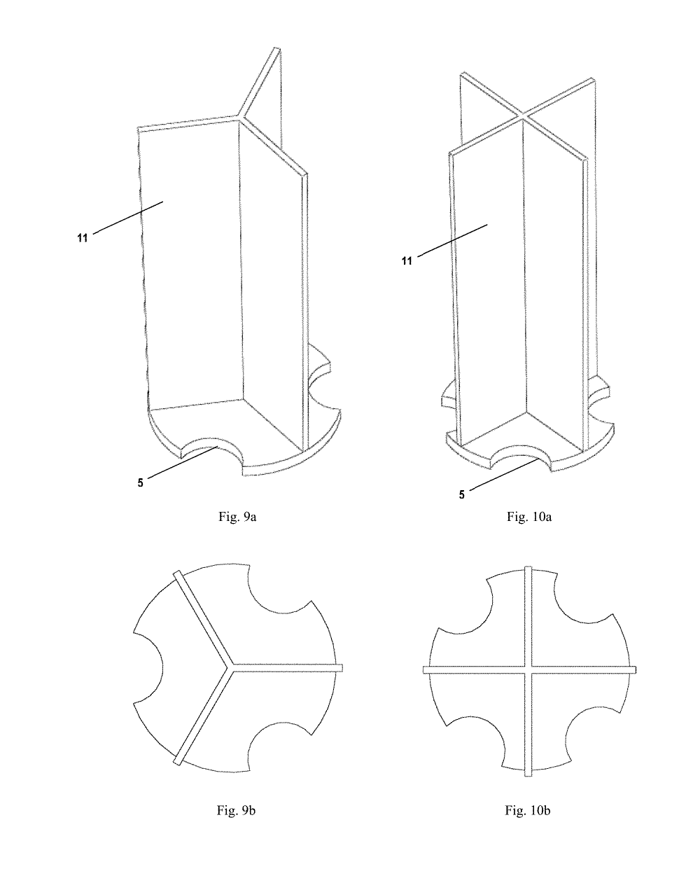

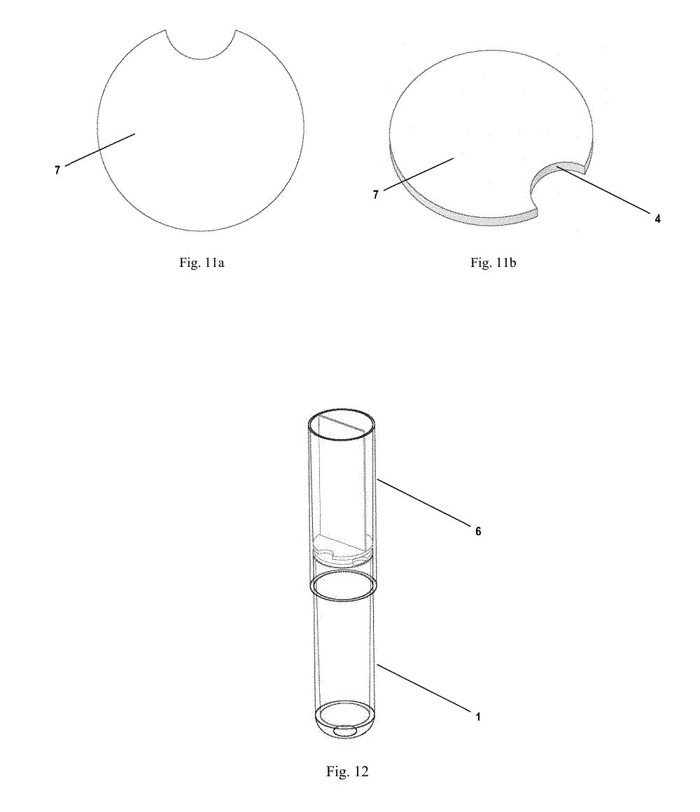

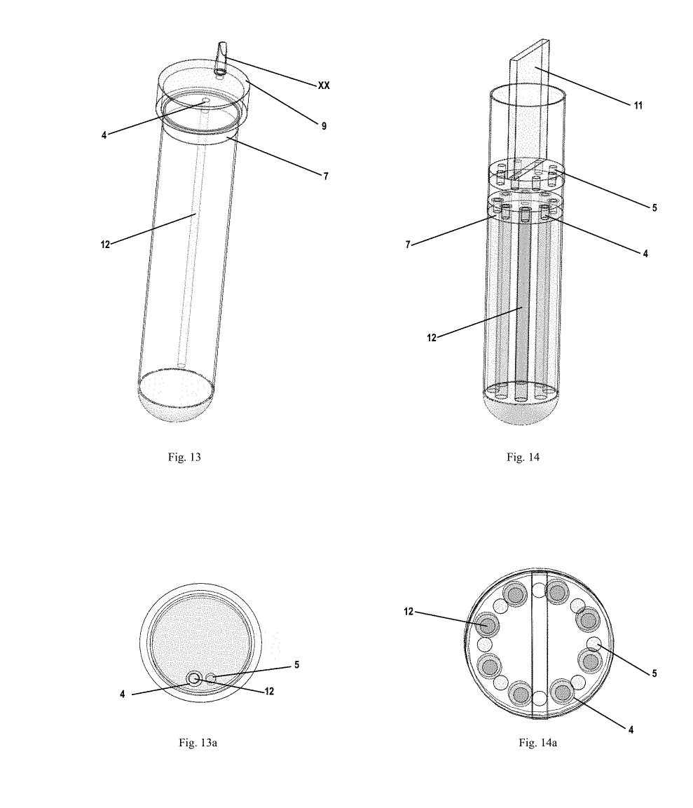

FIG. 1 illustrates a container in the shape of a centrifuge tube, intended for collecting fluids, especially biological material, it also illustrates the device which together with the container is used for density gradient liquids separation, according to the invention—the device enables layering of liquids in the container, prior centrifugation, one on top of another with maintaining clear interphase between them.

FIG. 2 and FIG. 3 illustrate respectively a longitudinal sectional view and a side view of a container in the shape of a centrifuge tube, wherein, for a better understanding of the invention—the discs that the partition is built of are spaced apart;

FIG. 4 and FIG. 5 illustrate respectively a side view and a longitudinal section of the tube-shaped container with visible narrowing of the inner diameter of the tube and with increasing wall thickness.

FIG. 6 illustrates a cross-section through the container-shaped tubes in the embodiment without vertical partition, and illustrates the air duct in the device partition,

FIG. 7a and FIG. 7b illustrate respectively a side view and cross-section of the upper part of the device in the form of a disk with incomplete vertical partition

FIG. 8a and FIG. 8b illustrate respectively a side view and cross-section of the upper part of the device in the form of a disk with vertical partition of rectangular shape,

FIG. 9a and FIG. 9b illustrate respectively a side view and cross-section of the upper part of the device with vertical partition build of three rectangles,

FIG. 10a and FIG. 10b illustrate respectively a side view and cross-section of the upper part of the device with vertical partition build of two intersecting rectangles forming a cross shape.

FIG. 11a and FIG. 11b illustrate a sectional and a side view of the partition disc with a cutout.

FIG. 12 illustrates one embodiment of the invention, wherein the device is fitted onto the container for centrifugation.

FIG. 13 and FIG. 13a shows the device in a sectional side and from top view, which allows fitting separate upper chamber on top the device with guide in a form of elongated cylinder,

FIG. 14 and FIG. 14a and illustrates the device in a sectional side and top view with a guide in the form of eight elongated cylinders.

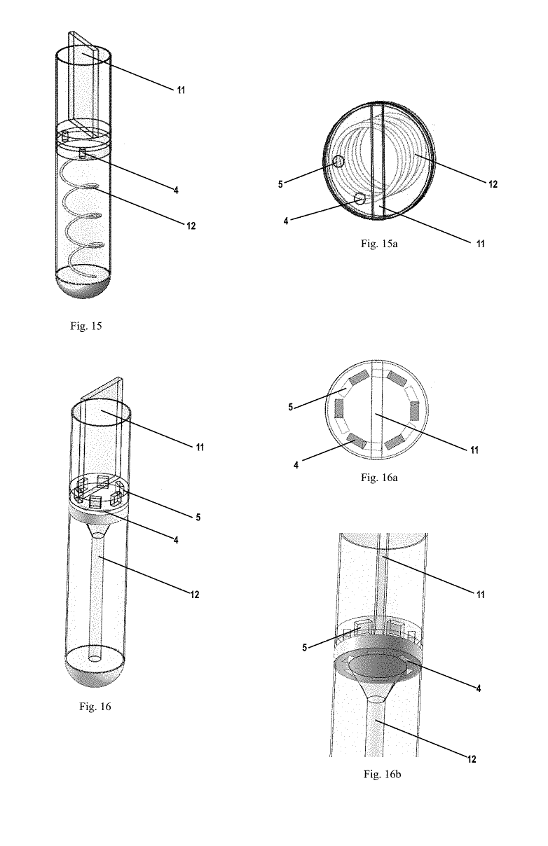

FIG. 15 and FIG. 15a shows the insert in a sectional side and top view equipped with a guide in the form of spiral,

FIG. 16 and FIG. 16a shows the insert in a sectional side and top view, equipped with a guide in a form of a funnel.

### EMBODIMENT 1

As illustrated on FIG. 1 in the first embodiment of this invention device 6 for the centrifuge container in a form of a centrifuge tube is built of a flat circular disc 7, tightly fitted to the inner walls of the tube partition, and another circular disc 8 which both 7 and 8 constitute the device partition and of a full vertical partition 11 which is attached to disc 8. The device in this embodiment of the invention is placed inside the centrifugation container 1 which is a centrifugation tube with 0.23″ diameter. The device 6 in this embodiment is made of plastic, but could also be made of other materials. A shown in FIG. 12 the device 6 can be placed in another container that can be fitted on to the centrifuge container 1, in this case device is outside of the centrifuge container 1.

In this embodiment inner walls of the centrifuge container 1 are at the same time the guide 12 and that centrifuge container walls thickens, inner diameter of the centrifuge container decreases gradually toward its' bottom. In this embodiment of the invention inner wall of the container 1 is the guide 12, which directs the down-flow of liquids from upper chamber 2 to the lower chamber 3 via the aperture 4. Liquids—in particular biological fluids being separated to fraction—flow down to the bottom of the container 1 on and along the guide 12—being in this embodiment the internal wall of the container 1—and liquids layer one on top of the another on the bottom of the container 1. Flow-down of liquids along or on the guide 12 prevents mixing of liquids, which otherwise would impair separation of these liquids.

In this embodiment of this invention partition 7 has shape of circular disc which in transverse section has shape of a circle (FIG. 11a, FIG. 11b) and its' shape is tightly fitted to the transverse section on the container 1, therefore the diameter of the partition is longer on the top side compared to the bottom side, and its' longitudinal section closely resembles the shape of flattened inverted trapezium. Partition 7 divides container 1 to upper chamber 2 and lower chamber 3. Partition in this embodiment has an aperture 4 which is a notch in the shape resembling semicircle.

As shown on FIGS. 8a and 8b, vertical partition 11 may have a shape of rectangle, which adheres tightly to the inner walls of the container 1, whereupon vertical partition 11 attached to the disc 8 separates upper chamber 2 of the container 1 in the shape of a tube to two sub-chambers 10a, 10b. In each half of the disc 8 shaped by the vertical partition 11 is one aperture 5 in a shape of a notch, which can be closed by disc 8. I this embodiment of the invention apertures 5 in a shape of a notch in disc 8 are in a shape of semicircle. In other embodiments of the invention it is possible to use discs 8 with apertures 5 in different shapes. The shape of apertures 4, 5 and their positioning against each other determines the speed of liquids down-flow from upper chamber 2 to lower chamber 3.

In this embodiment of the invention apertures 4, 5 are in shape of a semicircular notch with 0.115″ radius and have identical shape. In different embodiments of the invention apertures 4, 5 can have various shapes, and shapes can be different from one another, however their diameter should not be bigger than 0.1″. In such arrangement of the partition 7 and disc 8 that apertures 4, 5 are not overlapping, down-flow of liquids between upper chamber 2 and lower chamber 3 is blocked and flow of liquids cannot take place.

In this embodiment of the invention container 1 is equipped with lid 9. In one embodiment of the invention lid 9 has a gap, through which protrudes upper part of the vertical partition 11 of the device 6. Such location of the vertical partition 11 enables changes of the position of the disc 8 in relation to disc 7 by turning of the protruding part of the vertical partition 11 and at the same time movable part of the lid 9. Container 1 and lid 9 has a thread and is a nut. Alternatively lid without a gap 91 can be used, wherein vertical partition 11 of the device is adjusted to the length of the container 1 in such a way that after screwing down the lid 9 vertical partition 11 tightly adheres to the inner side of the lid 9. Lid 9 may be made of polymers and can have calibrated scale for turning/screwing the lid 9. On the container 1 for centrifugation and on the lid 9 labels may be present to facilitate correct adjusting/arranging of the apertures 4, 5 positions against each other.

Alternatively in different embodiments of the invention different shapes and positions of the vertical partition 11. As illustrated in FIGS. 7a and 7b, vertical partition 11 does not have to adhere to the inner walls of the container 1, in which case vertical partition 11 placed on disc 8 separates the tube only to two chambers—upper chamber 2 and lower chamber 3 and upper chamber 2 is not further divided to additional sub-chambers. In this embodiment of the invention, disc 8 is equipped in one aperture 5 in a shape of a notch, in the other embodiment of the invention shape of the disc 8 could be limited to the size that would enable closure of the apertures 4 in the disc 7.

As illustrated in the FIGS. 9a and 9b, vertical partition 11 can be built of three elements in the shape of a rectangle connected with each other with longer edges, which other edges adhere tightly to the inner wall of the container 1, in this embodiment vertical partition 11 placed on the disc 8 divides upper chamber 2 of the container 1 in the shape of the tube to three sub-chambers. In this embodiment, disc 8 has three notches 5, one in each of the sub-chambers.

As illustrated in FIGS. 10a and 10b, vertical partition 11 may be built of four rectangles connected with each other, which edges adhere tightly to the inner wall of the container 1, in this embodiment vertical partition 11 placed on the disc 8 divides upper chamber 2 of the container 1 in the shape of the tube to four sub-chambers. In this embodiment, disc 8 has four notches 5, one in each of the sub-chambers.

Device 6 may also be used in containers 1 shaped differently than centrifuge tube presented in this example of invention embodiment, however there has to be a method that allows to centrifuge this container.

### EMBODIMENT 2

FIGS. 13 and 13a show another embodiment of the invention, wherein the device 6 has a baffle 7, which does not have an upper chamber but allows the connection through a tube (see part 16) down to upper partition in a form of a container (for example, a test tube, pouch, bag) with separation medium or separation liquid. Subsequently, the partition is equipped with a guide 12 in a form of an elongated cylinder which is attached to the partition 7 and is situated at a distance from the aperture 4. This allows fluid flow from the upper chamber 16 through the tube followed by the aperture in the partition along the guide the lower chamber 3. In this embodiment, the elongated cylinder forms a guide 12 and its length is such that the test material spreads gently on a surface of the centrifugal medium used in the gradient separation method and it does not cause significant disturbances to the separation medium.

### EMBODIMENT 3

FIGS. 14 and 14a show another embodiment of the invention, wherein the insert 6 has a partition 7, equipped with a guide 12 in a form of eight elongated rollers which are anchored to partition 7 and are located at such distance from the aperture 4, which allows the liquid to flow from the upper chamber through the aperture, in the partition along the guide, to the lower chamber 3. In this embodiment, the length of the guide for the elongated rollers 12 is such that the test material spreads gently on a surface of the centrifugal medium used in the gradient separation method and it does not cause significant disturbances to the separation medium.

### EMBODIMENT 4

On the other hand, FIGS. 15 and 15a show yet another embodiment of the invention, wherein the device 6 has a partition 7 equipped a guide 12 in the shape of a spiral. In analogy to Example 2, the length of the coil should be such that the test material spreads gently on a surface of the centrifugal medium used in the gradient separation method and it does not cause significant disturbances to the separation medium.

### EMBODIMENT 5

FIGS. 16, 16a and 16b show yet another embodiment of the invention, wherein the insert 6 has a partition 7 provided with a guide 12 in the shape of a funnel. Wherein the four holes in the partition 7 directs the fluids from the upper chamber so as to roll down the outer surface of the funnel to the bottom of the lower chamber 3. In analogy to Example 2, the length of the coil should be such that the test material spread over a surface of the medium to the gradient centrifugation thereby causing no significant adverse to the separation medium.

### EMBODIMENT 6

Method for separation of fractions of given density from fluid sample with fractions of different density according to the invention can be achieved by, filling two sub-chambers 10a, 10b of the upper chamber 2 with two media for separation in on density gradient, first medium has density of 1.119 g/mL second medium has density of 1.077 g/mL (respectively Histopaque 1.119 and Histopaque 1.007 Sigma Aldrich), at the same time apertures 4, 5 being notches—respectively in disc 7 and disc 8—are not overlapping and remain in closed position. Next by changing the position of disc 8 by its' turning, apertures 4, 5 overlap each other in such a way that enables down-flow of mediums from the upper chamber 2 to the lower chamber 3. Down-flow occurs on and along the guide 12 which in this embodiment is the internal wall of the container 1. Media are added one by one starting from the highest density to the lowest density, and interface is established between media of different densities. Next to one of the empty sub-chambers 10, with closed down-flow between the upper chamber 2 and the lower chamber 3, fluid or mixture designated to be separated to fractions of different densities in density gradient centrifugation e.g. native or diluted blood.

The size of the clearance created by apertures 4, 5 being the notches of respectively disc 7 and disc 8 can be controlled by regulation of positions of disc 7 and disc 8 against each other. Slow turning of the upper part of the vertical partition 11, and subsequently disc 8, causes gradual increase of the down-flow velocity up to the moment when expected velocity, of liquid down-flow from the upper chamber 2 to the lower chamber 3, is achieved. By regulation of positions of disc 7 and disc 8 against each other, liquid down-flow can be controlled in order to achieve stable laminar flow of liquid on and along the internal wall 12 of the centrifuge container 1. Construction of discs 7 and disc 8 according to the invention ensures very gentle down-flow of the liquid from the upper chamber 2 to the lower chamber 3 of the centrifuge container 1 in such a way that the surface of the liquid is intact and subsequently added liquids which down-flows from the upper chamber 2 does not mix with the liquid already present in the lower chamber 3.

After stratified down-flow of the two liquids for separation on density gradient these liquids layer one on top of the another because of different density, analyzed sample was added—blood in this case—although it is possible to use different types of separation liquids, including native or diluted biological samples. Blood was first placed in sub-chamber 10a, and next after turning the disc 8 of the device 6 in such a way that aperture 4 of the disc 7 was overlapping at least partially with respective aperture 5 in the disc 8 of the device 6 and enables down-flow of the blood on and along the inner wall 12 of the container 1 from the sub-chamber 10a to the lower chamber 3 layering it on the surface of previously placed separation media. Because of the device 6 construction it is not necessary to place the biological material in the container 1 with extraordinary precision and care.

Next blood in lower chamber 3 of the container 1 is centrifuged according to methods known in the field. During centrifugation two directional flow of liquids occurs within different compartments created by separation liquids of different density in the lower chamber 3, at the end of centrifugation continuous density gradient establishes with red blood cells sedimenting to the bottom creating lowest placed layer, layer above is a liquid of 1.119 g/mL density, layer above is layer of polymorphonuclear cells, layer above is a liquid of 1.077 g/mL density, layer above is layer of peripheral blood mononuclear cells, layer above is the highest layer of plasma. After removing of the insert, each layer of cells/or fluid can be removed by aspiration with the use of a pipet or by decantation.

### EMBODIMENT 7

Insert and method of the invention is used, for example, for separating the desired subset of blood cells. In this embodiment of ten samples of blood were taken from healthy volunteers (20 ml of venous blood) to a commercially available tubes with versene acid (EDTA) (EDTA tube, Becton Dickinson). In this experiment, the volume of the centrifuge tube 1 of which the essence of the invention was 50 ml, was also used for the separation of two media of different densities (Histopaque 1119 and Histopaque 1077 Sigma Aldrich). For the separation of fluids used have a neutral pH, be isotonic to body fluids, the first separation medium to have a density of 1.119 g/ml, while the second had a density of 1.077 g/ml.

Then 10 ml of a medium provided for the separation of a density of 1.119 g/ml in sub-chamber 10a into the upper chamber 2 of the container 1 for centrifugation provided with the device 6 of the invention. A second fluid having a density of 1.077 g/ml with a volume of 10 ml was placed in sub-chamber 10b of the upper chamber 2, and then laminated imposed by the first medium by means of an insert 6 of the invention described above. In the experiment divider had a thickness of 0.08″ and the cutouts 4, 5, 7 and the baffle disc 8 have a radius of 0.115″. Then, the collected blood is versene acid (EDTA) provided in the upper sub-chamber 10a of the chamber 2. Each blood sample was applied to the surface layer media separation by the insert 6 of the invention described above.

In a further step, all tubes were centrifuged at 700 g (with minimal acceleration and without active braking) for 30 minutes at room temperature. In the process of density centrifugation, the blood was separated into four fractions: plasma, mononuclear white blood cells (PBMC), white blood cells with a segmented nucleus (PMN), and Czerwonki cells. Purity fraction of PBMC and PMN was confirmed by flow cytometry. Purity PBMC and PMN in the fractions was 95% and 92%. PBMC and PMN were undetectable in plasma fractions. Isolated plasma, PBMC and PMN were suitable for further analysis, including, but not limited to aPatryk, analysis: RNA, micro-RNA, mitochondrial DNA, nuclear DNA, proteins and phenotyping of the cells.

1. A device for a centrifugation container, for separation of liquid fractions having a desired density range, for biological and/or liquids forming suspensions, the device comprising: a partition that separates an interior of the container into at least two chambers in a vertical arrangement—an upper chamber and a lower chamber, and the device having the partition has an aperture lined up with a guide, on which a fluid sample, can flow down from an upper chamber to a lower chamber, of the container for centrifugation.

2. The device according to claim 1, wherein the guide is an inner wall of centrifuge container, a spiral, funnel or vertical elements in the shape of an elongated cylinder.

3. The device according to claim 1, wherein the partition consists of two adjacent surfaces with apertures, in the shape of flattened disks fitted to a container having a cross section similar to the wheel, wherein the surfaces are movable with respect to each other and positioning relative to each other can be adjusted allowing for closing communication via partition apertures.

4. The device according to claim 1, wherein the upper chamber further comprises a vertical partition or partitions dividing the upper chamber into sub-chambers each of the sub-chambers having an apertures.

5. A container for centrifugation comprising device for centrifugation container, for separation of liquid samples having a desired density range, forming a suspension or biological fluids, the device has a partition that separates an interior of the container into an upper chamber and a lower chamber, and the partition has an aperture and near the aperture is a guide along which the down-flow of separation liquids flow to the lower chamber of the centrifugation container.

6. A method for separating out a fraction having a desired density range from a biological sample containing fractions of different density, comprising:
a) providing a container with the device for the centrifuge container, for separation of liquid sample to fractions having a desired density range by density gradient centrifugation, the liquid sample being suspensions or biological fluids,
b) filling a lower chamber of the container with medium for density gradient separation, or an upper chamber of the container is filled with the medium, which then through an aperture in a device partition flows down on a guide to the lower chamber;
c) pouring the fluid sample designated to be separated to fractions of different densities into the lower chamber, by filling the upper chamber or at least one of upper sub-chambers or by attaching the upper chamber to the device partition so that fluid sample flows down to the lower chamber through the aperture in the device partition and then along the guide and layer on the surface of separation liquids already present in the lower chamber, maintaining an interphase between liquids;
d) centrifuging the separation container until the sample separates into fractions of different density.

7. The method according to claim 6, wherein step (b) is followed by an additional step or steps (b) which entails addition of an additional medium for density gradient separation, additional media are added in order from highest to lowest density.

8. The method according to claim 6, wherein after step (d) selected fractions of different density from separated liquid sample can be studied, tested and analyzed, these fractions are preserved by freezing.

9. The method according to claim 6, wherein in case of separating blood to fractions of different density, each separated fraction (with different) density contains different blood elements including: leukocytes, platelets, erythrocytes, bone marrow cells, cells suspended in homogenate including endothelial cells, neurons, fungus, viruses, microparticles including exosomes, cellular fragments, cell organelles including nuclei, mitochondria, chloroplasts.

10. A kit comprising:
a) the device for a container for centrifugation, for separation of liquid sample to fractions of different density by density centrifugation, liquids of the liquid sample forming a suspension or comprising biological fluids, wherein the device has a partition dividing an interior of the container into an upper chamber and a lower compartment, wherein the partition has an aperture with a guide, along which the liquid sample flows, to the lower chamber of the container for centrifugation
b) at least one medium for density gradient separation.
